(19) United States
(12) Plant Patent Application Publication
(10) Pub. No.: US 2013/0263345 P1
(43) Pub. Date: Oct. 3, 2013

(54) Impatiens plant named "DHAR318"

(50) ;

(71) Applicant: Gavriel Danziger, Moshav Mishmar Hashiva (IL)

(72) Inventor: Gavriel Danziger, Moshav Mishmar Hashiva (IL)

(21) Appl. No.: 13/506,198

(22) Filed: Apr. 2, 2012

(51) Int. Cl. A01H 5/00

(52) U.S. Cl. PLT/318.3

(57) ABSTRACT

A new and distinct Impatiens cultivar named ‘DHAR318’ is disclosed, characterized by distinctive large salmon flowers, good branching and sun resistance. The new variety is an Impatiens normally produced as an outdoor ornamental plant for containers or gardens.

### LATIN NAME OF THE GENUS AND SPECIES

Impatiens hawkeri

### VARIETY DENOMINATION

‘DHAR318’

### BACKGROUND OF THE INVENTION

The new Impatiens cultivar is a product of chance discovery. The variety originated as a naturally occurring mutation of the variety Impatiens hawkeri ‘DHAR319’, filed concurrently for United States Plant patent.

The new variety was discovered by the inventor, Gavriel Danziger, a citizen of Israel, during March of 2009 in a research greenhouse in Moshav Mishmar Hashiva, Israel. After identifying the new variety as a potentially interesting selection, the inventor continued confidential testing and propagation of ‘DHAR318’, assessing stability of the unique characteristics of this variety.

Asexual reproduction of the new cultivar ‘DHAR318’ by leaf cuttings was first performed at a research greenhouse in Moshav Mishmar Hashiva, Israel in March of 2009 and has shown that the unique features of this cultivar are stable and reproduced true to type in successive generations.

### SUMMARY OF THE INVENTION

The cultivar ‘DHAR318’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DHAR318’ These characteristics in combination distinguish ‘DHAR318’ as a new and distinct Impatiens cultivar:

1. Large flowers.

2. Salmon flower color.

3. Good branching.

4. Sun resistance.

Plants of the new cultivar ‘DHAR318’ are similar to plants of the parent, Impatiens ‘DHAR319’, filed concurrently, in most horticultural characteristics, however, plants of the new cultivar ‘DHAR318’ produce salmon colored flowers, whereas ‘DHAR319’ produces purple colored flowers.

### COMMERCIAL COMPARISON

Plants of the new cultivar ‘DHAR318’ are comparable to the variety Impatiens ‘SAKIMP008’, U.S. Plant Pat. 21,014. The two Impatiens varieties are similar in most horticultural characteristics however, the new variety ‘DHAR318’ differs in having darker colored flowers, and a more vigorous growth habit. Additionally, the new variety has larger flowers than ‘SAKIMP008’.

Plants of the new cultivar ‘DHAR318’ can also be compared to the commercial variety Impatiens ‘SAKIMP016’, U.S. Plant Pat. 21,753. These varieties are similar in most horticultural characteristics, however ‘DHAR318’ has a more vigorous growth habit and larger flower size. Also ‘DHAR318’ has a darker colored flower than that of ‘SAKIMP0111’.

### BRIEF DESCRIPTION OF THE PHOTOGRAPHS

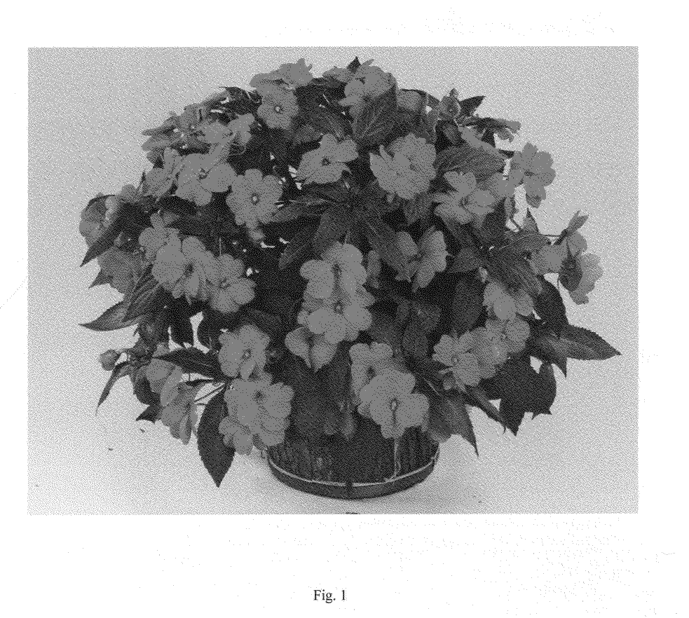

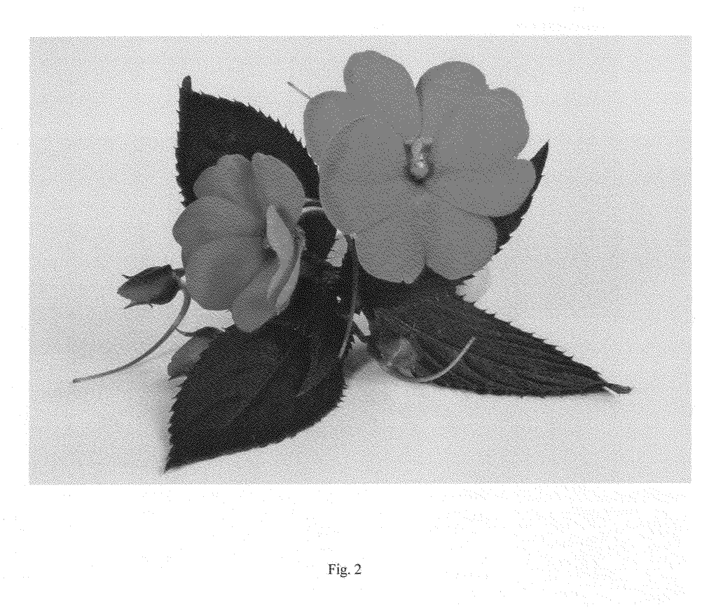

The accompanying photograph in FIG. 1 illustrates in full color a typical plant of ‘DHAR318’ grown in a greenhouse, in a 16 cm pot. Age of the plant photographed is approximately 14 weeks from a rooted cutting. FIG. 2 illustrates in full color a close up of a typical bloom of ‘DHAR318’. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2005, except where general terms of ordinary dictionary significance are used. The following observations and measurements describe ‘DHAR318’ plants grown in a greenhouse during Winter and Spring in Moshav Mishmar Hashiva, Israel. The growing temperature ranged from 20° C. to 35° C. during the day and from 15° C. to 25° C. during the night. General light conditions are bright, normal sunlight. Measurements and numerical values represent averages of typical plant types.
Botanical classification: Impatiens hawkeri ‘DHAR318’.
Propagation:
   Time to initiate roots.—Summer: 10 to 12 days at approximately 25° C. Winter: 14 to 20 days at approximately 18° C.
   Root description.—Fibrous adventitious roots.
Plant:
   Growth habit.—Mounded.
   Pot size of plant described.—16 cm.
   Age of plant described.—Approximately 14 weeks from rooted cutting.
   Height.—28 cm.
   Plant spread.—50 cm.
   Growth rate.—Vigorous.
   Branching characteristics.—Highly branched.
   Length of primary lateral branches.—25 cm.
   Quantity of primary lateral branches.—6-8.
   Characteristics of primary lateral branches.—Form: oval shape Diameter: 1 cm. Color: Yellow Green group 148 C RHS Texture: Smooth Strength: Strong Internode length: 6 cm.
Foliage:
   Leaf.—Arrangement: Alternate. Quantity: Approximately 20-25 per branch. Average Length: 7.5 cm. Average Width: 3 cm. Shape of blade: Lanceolate to ovate Apex: Attenuate. Base: Acuminate Margin: Ciliate Texture of top surface: Smooth Pubescence: none Aspect: 45 degrees Color: Young foliage upper side: Green Group 137 A RHS Young foliage under side: Green Group 138 B RHS Mature foliage upper side: Yellow Green Group 147 A RHS Mature foliage under side: Yellow Green Group 148 C RHS Venation: Midrib with secondary veins. Pinnate, only lower surface Type: Pinnate. Venation color upper side: Yellow Green Group 145 C RHS Venation color under side: Greyed Red Group 182 B RHS Petiole: Length: 0.8 cm. Diameter: 0.2 cm. Color: Red Group 51 D RHS Texture: Smooth.
Flower:
   Natural flowering season.—Flowers at any day length. All year round in a greenhouse environment. Flowering is continuous from spring to fall in an outdoor garden.
   Days to flowering from rooted cutting.—10 weeks.
   Inflorescence and flower type and habit.—Single flowers raised above the foliage. The corolla consists of five petals.
   Rate of flower opening.—10 to 15 days from bud to fully opened flower.
   Flower longevity on plant.—5-8 Days.
   Persistent or self-cleaning.—Self cleaning.
   Bud.—Shape: Ovate Length: 2 cm Diameter: 1 cm Color: Red Group 43 B RHS.
   Flower size.—Diameter: 8 cm. Depth: 0 cm.
   Petals.—Quantity: 5 Arrangement: Rotate overlapping Length: 4 cm Width: 4.5 cm Shape: Obcordate Apex: Heart Shape Base: Acute Margin: Entire Texture, upper and lower surfaces: Smooth.
   Color.—Salmon. When opening, upper surface: Red Group 43 B RHS When opening, lower surface: Red Group 41B RHS Fully opened, upper surface: Red Group 43 B RHS Fully opened, lower surface: Red Group 41B RHS.
   Spurs.—Quantity per flower: 1 Length: 6.5 cm Diameter: 0.1 cm Aspect: Spur curvature 40°-45° from peduncle Color: Towards base: Red Group 51A RHS Apex: Yellow Green Group 149 D RHS.
   Sepals.—Quantity: 2 Length: 2 cm Width: 1 cm Shape (overall): Oblong Apex: Acute Base: Rounded Margin: Entire Color: Yellow Green group 146 D RHS.
   Peduncles.—Length: 6 cm Diameter: 0.2 cm Angle: About 45° to the lateral branch Strength: Stable, flexible Texture: Smooth Color: Yellow Green Group 144 C RHS.
   Fragrance.—None.
Reporductive organs:
   Stamens.—There are five stamens with introrse anthers that are more or less fused and form a cap over the ovary. Number: 5.
   Anthers.—Shape: Oblong Length: Approximately 0.4 cm. Color: Red Purple Group 67 A RHS Pollen: Color: White Group 155 D RHS Quantity: Abundant.
   Pistil.—Number: 1 Length: 0.5 cm. Style: Length: 0.5 cm. Color: Yellow Green Group 143 A RHS Stigma: Shape: Round Color: White GROUP N 155B RHS Ovary Color: Yellow-Green group, RHS 144D RHS.
Other characteristics:
Seeds and fruits: Minute locule fruits produce 5-10 seeds per fruit.
   Disease/pest resistance: Neither resistance nor susceptibility has been observed.
Temperature tolerance: Grows best at a range between 10° C.-30° C. Will tolerate moderate temperatures outside of this range.

What is claimed is:

1. A new and distinct cultivar of Impatiens plant named ‘DHAR318’ as herein illustrated and described.
